(19) United States
(12) Patent Application Publication
(10) Pub. No.: US 2024/0288394 A1
(43) Pub. Date: Aug. 29, 2024

(54) DETECTION DEVICE

(71) Applicant: TAIYO YUDEN CO., LTD., Tokyo (JP)

(72) Inventors: Hisashi MAKI, Tokyo (JP); Kazuki SATO, Tokyo (JP)

(73) Assignee: TAIYO YUDEN CO., LTD., Tokyo (JP)

(21) Appl. No.: 18/589,291

(22) Filed: Feb. 27, 2024

(30) Foreign Application Priority Data
Feb. 28, 2023 (JP) 2023-030277

(51) Int. Cl. G01N 27/22; G01N 27/02; G01N 27/12; G01N 33/00

(52) U.S. Cl. CPC G01N 27/227; G01N 27/021; G01N 27/12; G01N 33/0031

(57) ABSTRACT

A detection device includes a plurality of sensors each of which is configured to detect one or more substances causing an odor in a gas, a storage chamber that stores the plurality of sensors, one or more sensor substrates each forming at least a part of a lower surface of the storage chamber and each having an upper surface on which one or more of the plurality of sensors are mounted, and a cover provided on the one or more of sensor substrate, having one or more openings that expose the plurality of sensors, and being in contact with the upper surface of the one or more sensor substrates.

### FIELD

A certain aspect of the present disclosure relates to a detection device.

### BACKGROUND

A detection device for detecting information about gas such as a smell is known (for example, Patent Documents 1 and 2). It is known that a part of a surface forming a flow path through which the gas flows is formed by a substrate on which a plurality of sensors are mounted (for example, Patent Document 1).

### PRIOR ART DOCUMENTS

### Patent Document

[Patent Document 1] International Publication No. WO 2021/172592
[Patent Document 2] Japanese Laid-open Patent Application Publication No. 2020-193846

### SUMMARY OF THE INVENTION

The substrate on which the plurality of sensors are mounted has a low thermal conductivity. Therefore, a temperature difference between the plurality of sensors becomes large. This reduces the detection accuracy of information about the gas and the like.

In view of the above problems, an object of the present disclosure is to suppress the temperature difference between the plurality of sensors.

(1) According to a first aspect of the embodiments, there is provided a detection device including: a plurality of sensors each of which is configured to detect one or more substances causing an odor in a gas; a storage chamber that stores the plurality of sensors; one or more sensor substrates each forming at least a part of a lower surface of the storage chamber and each having an upper surface on which one or more of the plurality of sensors are mounted; and a cover provided on the one or more of sensor substrate, having one or more openings that expose the plurality of sensors, and being in contact with the upper surface of the one or more sensor substrates.

(2) In the above configuration (1), a plurality of sensor substrates may be provided. The plurality of sensor substrates may be adjacent to each other, and the cover may be in contact with the upper surface of each of the plurality of sensor substrates.

(3) In the above configuration (2), the cover may be provided on a peripheral edge region of the upper surface of each of the plurality of sensor substrates.

(4) In the above configurations (2) or (3), the detection device further may include a circuit substrate electrically connected to the plurality of sensor substrates and being provided below the plurality of sensor substrates. The plurality of sensor substrates may be detachably attachable to the circuit substrate.

(5) In any one of the above configurations (1) to (3), at least some of the one or more openings may expose two or more of the plurality of sensors.

(6) In the above configuration (5), a shortest distance between each of the plurality of sensors and a side surface of the opening may be smaller than a maximum width of each of the plurality of sensor.

(7) In any one of the above configurations (1) to (3), a conductor pattern may be provided on the upper surface of the one or more sensor substrates, and at least a lower surface of the cover may have an insulation property, or an insulating layer may be provided between the cover and the conductor pattern.

(8) In the above configuration (7), the conductor pattern may be a pad provided around at least one of the plurality of sensors or an element constituting a part of a circuit electrically connected to the at least one of the plurality of sensors.

(9) In any one of the above configurations (1) to (3), a thermal conductivity of at least a part of layers in the cover may be higher than a thermal conductivity of an insulating layer in the one or more sensor substrates.

(10) In any one of the above configurations (1) to (3), the storage chamber may be defined by a housing, the one or more sensor substrates and the cover. The housing may have a lower wall portion an upper surface of which forms a part of the lower surface of the storage chamber, the lower wall portion of the housing may have a cavity, the one or more sensor substrates may be provided inside the cavity in a plan view, and the cover may be mounted to the lower wall portion via a mounting member. A difference in height between the upper surface of the one or more sensor substrates and the upper surface of the lower wall portion may be 1/10 or less of a thickness of the cover.

(11) In any one of the above configurations (1) to (3), a thickness of the cover may be ½ times or more and twice or less a thickness of the plurality of sensors.

(12) In the above configuration (2) or (3), the detection device further may include an environment sensor substrate having an environment sensor provided on an upper surface thereof, the environment sensor being a temperature sensor or a humidity sensor. The cover may be in contact with the upper surface of each of the plurality of sensor substrates and the upper surface of the environment sensor substrate, and the one or more openings may expose the plurality of sensors and the environment sensor.

### BRIEF DESCRIPTION OF THE DRAWINGS

FIG. 1 is a cross-sectional view of a detection device according to a first embodiment;

FIG. 2 is a cross-sectional view of a detection device according to a first comparative example;

FIG. 3 is a cross-sectional view of a detection device according to a second embodiment;

FIG. 4 is a plan view of the detection device according to the second embodiment;

FIG. 5 is a plan view of the detection device according to the second embodiment;

FIG. 6 is a plan view of the detection device according to the second embodiment;

FIG. 7 is a schematic diagram of a sensor according to the second embodiment;

FIG. 8 is a block diagram of the detection device according to the second embodiment;

FIGS. 9A and 9B are diagrams illustrating the temperature of the sensor with respect to time in Experiment 1;

FIG. 10 is a plan view of another example of a detection device according to the second embodiment;

FIG. 11 is an enlarged plan view of the detection device according to the second embodiment;

FIGS. 12A to 12C are enlarged cross-sectional views of the detection device according to the second embodiment;

FIGS. 13A to 13C are enlarged cross-sectional views of the detection device according to the second embodiment; and

FIG. 14 is an enlarged cross-sectional view of the detection device according to the second embodiment.

### DETAILED DESCRIPTION

Embodiments of the present disclosure will be described below with reference to the drawings.

### First Embodiment

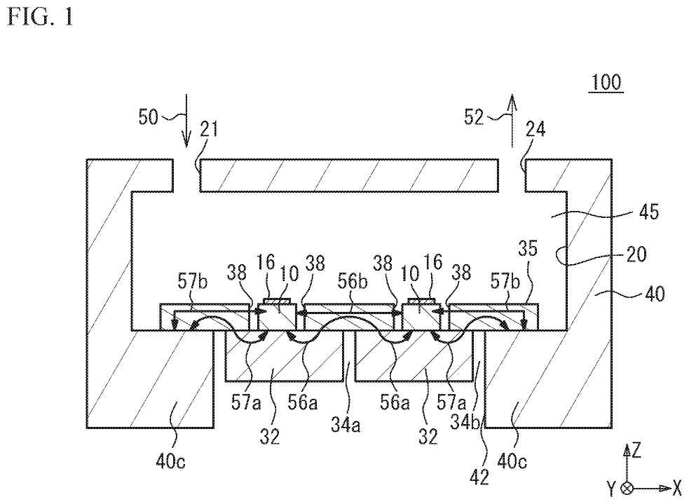

FIG. 1 is a cross-sectional view of a detection device 0 according to a first embodiment. The detection device 100 includes a housing 40, a plurality of sensor substrates 32, a plurality of sensors 10, and a cover 35. The housing 40, the sensor substrates 32, and an inner wall of the cover 35 form a space 45. The space 45 surrounded by the housing 40, the sensor substrate 32 and the cover 35 is a storage chamber 20 for storing the plurality of sensors 10. Each sensor 10 is an odor sensor for detecting one or more substances that cause an odor in a gas, which will be described later. The plurality of sensors 10 are mounted on the plurality of sensor substrates 32, respectively. This is to facilitate replacement of the sensor 10 by replacing the entire sensor substrate 32 when replacing the sensor 10.

The storage chamber 20 forming the space 45 has an upper surface, side surfaces connected to the upper surface, and a lower surface connected to the side surfaces. The upper surface and the side surfaces of the storage chamber 20 are an inner wall of the housing 40. The lower surface of the storage chamber 20 includes the inner wall of the housing 40, an upper surface of the cover 35, and upper surfaces of the plurality of sensor substrates 32, and a part of the sensor 10 is exposed. Here, only a part of the sensor 10 may be exposed in the space 45 without exposing the upper surface of the sensor substrate 32 in the space 45. The housing 40 has a lower wall portion 40c having a part of the lower surface of the storage chamber 20 as an upper surface and having the inner wall of the housing 40 as side surfaces. The lower wall portion 40c of the housing 40 has an opening 42. The opening 42 is a cavity. The sensor substrate 32 is provided inside the opening 42 so that at least a part of the sensor 10 is exposed to the storage chamber 20. The sensor substrate 32 is connected to a circuit substrate 31 via pins 33 as described later. Since the opening 42 is formed to replace the sensor substrate 32, the sensor substrate 32 may be provided on the upper surface or the side surfaces of the lower wall portion 40c without providing the opening 42 when replacement is not required.

The cover 35 is provided on the lower wall portion 40c of the housing 40 and the sensor substrates 32. The cover 35 has openings 38. At least a sensitive film 16 of the sensor 10 is exposed from the opening 38 to the space 45. The lower surface of the cover 35 is in contact with the lower wall portion 40c and the upper surface of the sensor substrate 32. At least a part of the cover 35 may be in contact with the upper surface of the sensor substrate 32. A gas 50 to be detected is introduced into the space 45 through an introduction path 21, and a gas 52 is discharged from the space 45 through a discharge path 24.

### Summary of Embodiment

The sensitive film 16 adsorbs and desorbs one or more substances that cause an odor. As a result, the electrical characteristics of the sensor 10, such as an oscillation frequency, resistance or capacitance, change. By measuring a change in electrical characteristics, it is possible to detect the one or more substances that cause the odor. However, the amounts of adsorption and desorption of the one or more substances to and from the sensitive film 16 varies with a temperature. The temperature also changes the electrical characteristics of the sensor 10, resulting in a change in the measured value. For example, in a sensor that measures the oscillation frequency of the sensor 10, the oscillation frequency of the sensor 10 changes due to the temperature.

The gas flowing into a measurement space is not uniform, resulting in a concentration distribution and a temperature distribution in the gas. If the temperature of the gas changes, a relative humidity changes, so if the gas has the temperature distribution, a distribution is generated in the relative humidity of the gas.

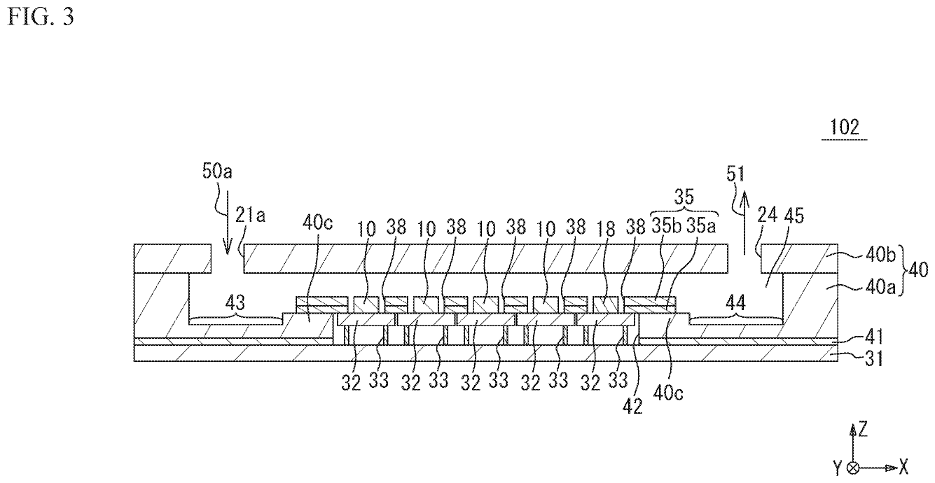

The temperatures of the sensor 10, the sensor substrate 32 on which the sensor 10 is mounted, and a mother substrate (e.g., the circuit substrate 31 in FIG. 3) located below the sensor substrate 32 also increase due to the operation. Components constituting a power supply and an oscillation circuit are mounted on the sensor substrate 32 or the circuit substrate 31, and heat is more remarkably generated in a part of the sensor substrate 32 or the circuit substrate 31. Further, when the size of the sensor substrate 32 increases and a distance between the adjacent sensors 10 increases, it becomes difficult to set the respective sensors 10 to the same temperature.

To improve the detection accuracy of the sensor 10, a humidity sensor and/or a temperature sensor (hereafter referred to as a sensor 18. See FIGS. 3 to 6) are located in the vicinity of the sensor 10 to correct an output signal of the sensor 10 using humidity and/or temperature. This improves the detection accuracy of the sensor 10. As with the temperature difference between the sensors 10, it is preferable that the temperature difference between the sensors 18 and 10 is small.

In view of the above, it is required to make the temperatures of the plurality of sensors 10 uniform. In the embodiment, the cover 35 having an excellent thermal conductivity is employed to solve this problem.

Description of cover 35: The cover 35 is a sheet-like or plate-like member having at least a front surface (upper surface) and a back surface (lower surface). The cover 35 may be made of a material having the excellent thermal conductivity, and is a metal layer, a ceramic layer, a resin layer, or a composite layer thereof. The cover 35 may not have the excellent thermal conductivity in some layers, for example, in order to secure an insulation property with respect to the sensor substrate 32 or to fix the cover 35 to the housing 40. As the material of the ceramic layer or the resin layer, it is preferable to adopt a material having a thermal conductivity close to that of a metal. In the case of the cover 35 made of a metal, the lower surface of the cover 35 is insulated or a thin insulating sheet is inserted between the cover 35 and the sensor substrate 32 in consideration of a short circuit with a conductor pattern of the sensor substrate 32.

Aspects of cover 35: The cover 35 includes the opening 38 to expose the sensitive film 16 of the sensor 10 to the measurement space 45. The upper surface of the sensitive film 16 is flush with the upper surface of the cover 35, is slightly recessed from the upper surface of the cover 35 or is slightly protruded from the upper surface of the cover 35. The cover 35 covers at least the plurality of sensor substrates 32. At least a part of the upper surface of the sensor substrate 32 and at least a part of the lower surface of the cover 35 are in contact with each other. The cover 35 covers the sensor substrate 32 on which the sensor 10 is mounted and the sensor substrate 32 on which the sensor 18 is mounted, and at least a part of the upper surface of the sensor substrate 32 on which the sensor 18 is mounted and at least a part of the lower surface of the cover 35 are in contact with each other.

Heat can be easily transmitted between the sensor substrates 32 through the cover 35. This makes the temperature of the gas in the vicinity of the sensor substrate 32 in contact with the cover 35 and the temperature of the gas inside the opening 38 of the cover 35 uniform. The gas having a uniformed temperature is supplied to the sensitive film 16 of the sensor 10. In particular, the sensor 10 is exposed from the opening 38 of the cover 35. Therefore, the temperature of the gas supplied to the sensors 10 becomes substantially uniform, and the temperature difference between the sensors 10 reduces. In addition, the temperature difference between the sensor 10 and the sensor 18 reduces.

(Sensor Substrate 32 on which Sensor 10 is Mounted)

The sensor substrate 32 is generally a printed circuit substrate, and is, for example, a resin substrate such as an epoxy-based or imide-based resin substrate, a ceramic substrate such as alumina or beryllia, or a metal substrate or a semiconductor substrate such as silicon, the upper surface of which is subjected to insulation treatment. In the sensor substrate 32, at least one layer of the conductor pattern is provided on the upper surface, and an insulating film is coated on the upper surface of the sensor substrate 32 to prevent the conductor pattern from oxidizing. The insulating film is, for example, an epoxy-based solder resist. When the sensor substrate 32 is the semiconductor substrate, the insulating film is a silicon oxide film, a silicon nitride film, a glass film, or the like, in addition to the solder resist. Therefore, the uppermost surface of the sensor substrate 32 is covered with a film serving as a passivation film. In some cases, the uppermost surface of the sensor substrate 32 may be a metal shield film to which a ground potential is supplied.

### (Reduction of Temperature Difference Between Sensors)

The sensor 10 and the sensor substrate 32 electrically operate, and therefore generate heat. The amount of heat generated varies greatly depending on the size of a circuit including the sensor 10 and the sensor substrate 32 and the magnitude of a current flowing through the circuit. However, if the cover 35 is in contact with the uppermost surface of the sensor substrate 32, heat generated in the sensor 10 and the sensor substrate 32 is conducted to the cover 35. Since the cover 35 has a high thermal conductivity, the temperature inside the cover 35 is substantially uniform. This can reduce the temperature difference between the sensors 10 and also reduce the temperature distribution of the gas in the vicinity of the sensors 10.

The contact between the cover 35 and the sensor substrate 32 includes the following aspects. When the uppermost surface of the sensor substrate 32 is the insulating film, the lowermost surface of the cover 35 may be a metal or an insulator, and the lower surface of the cover 35 is in contact with the insulating film on the uppermost surface of the sensor substrate 32. When a part of the uppermost surface of the sensor substrate 32 is a conductor, the lowermost surface of the cover 35 is an insulator, and the lower surface of the cover 35 is in contact with the conductor on the uppermost surface of the sensor substrate 32.

### First Comparative Example

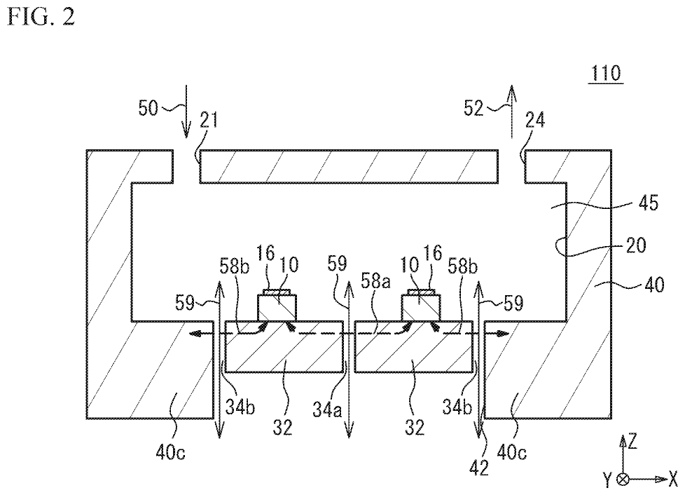

FIG. 2 is a cross-sectional view of a detection device 110 according to a first comparative example. The configuration of the second embodiment is the same as that of the first embodiment except that the cover 35 is not provided.

Subjects in the comparative example will be described below.

### (Subject 1)

In the detection device 110, heat is conducted between the sensors 10 via the sensor substrate 32 as indicated by a broken line arrow 58a in FIG. 2. The material of the insulating layer of the sensor substrate 32 is, for example, glass epoxy resin, and has a low thermal conductivity. If the sensor substrate 32 is provided for each sensor 10, a gap 34a is formed between the sensor substrates 32, which makes it harder to conduct heat. Therefore, heat exchange between the sensors 10 is not performed, and the temperature difference between the sensors 10 increases. Therefore, when the concentration of the odor or one or more specific substances is calculated based on the outputs of the plurality of sensors 10, the detection accuracy of the information about the gas reduces.

### (Subject 2)

The heat conduction between the sensors 10 and the housing 40 is performed through the sensor substrates 32 and the housing 40 as indicated by broken arrows 58b in FIG. 2. However, gaps 34b or an insulating material may exist between the sensor substrate 32 and the lower wall portion 40c, i.e., at contact portions. In this case, the heat conduction between the housing 40 and the sensor substrates 32 becomes difficult, and the temperature difference between the sensors 10 and the temperature difference between the housing 40 and the sensors 10 increase.

### (Subject 3)

In the detection device 110, the gaps 34a and 34b are formed. Therefore, as indicated by arrows 59 in FIG. 2, the gas in the storage chamber 20 and the gas below the sensor substrates 32 flow in and out through the gaps 34a and 34b depending on a temperature relationship between both gases. For example, when the mother substrate is provided below the sensor substrates 32, the temperature of the gas in the vicinity of the mother substrate increases due to a power supply circuit provided on the mother substrate. In this case, the gas in the space below the sensor substrate 32 flows into the storage chamber 20. Such inflow and outflow of gas disturbs the temperature of the gas and the concentration of the one or more substances that cause the odor in the vicinity of the sensor 10, resulting in a decrease in measurement sensitivity.

### (Subject 4)

The sensor substrate 32 is an organic insulator such as a glass epoxy resin, and the uppermost surface thereof is covered with an organic coating film such as an epoxy-based solder resist. These organic substances adsorb or desorb substances and moisture that cause the odor. This phenomenon of adsorption and desorption increases or decreases with temperature, and disturbs the temperature of the gas and the concentration of the substance that causes the odor in the vicinity of the sensor 10 in the storage chamber 20.

### (Description of Measures by First Embodiment)

(Arrangement of Sensor Substrates 32 on which the Sensors 10 and 18 are Mounted)

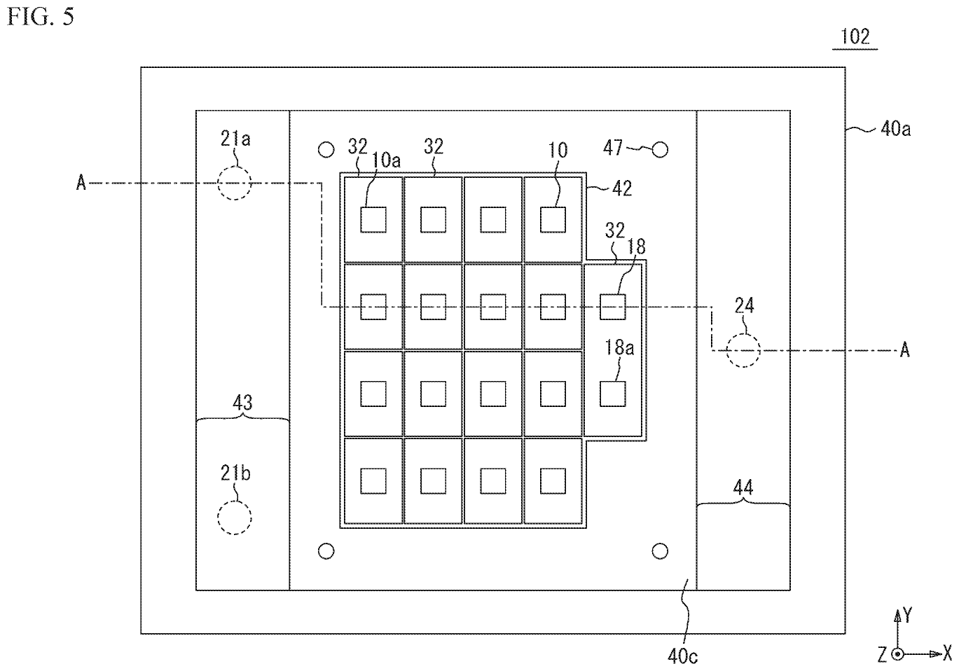

As illustrated in FIG. 5, since the sensors 10 are the same in size, the sensor substrates 32 on which the sensors 10 are mounted, respectively, are all substantially the same in size and rectangular in shape. The sensor substrate 32 on which the sensor 18 is mounted is preferably the same in size as the sensor substrate 32 on which the sensor 10 is mounted, but in FIG. 5, the sensor substrate 32 on which the sensor 18 is mounted is about twice the size of the sensor substrate 32 on which the sensor 10 is mounted. The sensor substrate 32 on which the sensor 18 is mounted and the sensor substrate 32 on which the sensor 10 is mounted are substantially the same in size in the X direction.

When the sensor substrates 32 are arranged in an orderly manner as in the case of a building block system in which cards having the same size are arranged in a vertical and horizontal direction, the sensor substrates 32 are designed so that the gaps 34a are not formed between the sensor substrates 32. For example, the sensor substrates 32 are arranged in a matrix. However, the gaps 34a may be formed between the sensor substrates 32 due to a manufacturing error of the sensor substrates 32.

### (Measures 1 and 2)

In the detection device 100 of the first embodiment, at least a part of the lower surface of the cover 35 is in contact with a part of the upper surface of each of the plurality of sensor substrates 32. As a result, as indicated by an arrow 56a in FIG. 1, heat is conducted between the sensor 10 in a surface direction through the sensor substrate 32 and the cover 35. Further, as indicated by an arrow 56b, heat is conducted between the sensors 10 through the gas in the openings 38 of the cover 35 without through the sensor substrate 32. Therefore, the detection sensitivity can be improved. As indicated by arrows 57a, the sensors 10 and the housing 40 conduct heat in the surface direction through the sensor substrates 32 and the cover 35. As indicated by arrows 57b, heat is conducted between the sensors 10 and the housing 40 through the gas in the openings 38 of the cover 35 without through the sensor substrates 32. Therefore, the detection sensitivity can be improved.

### (Measure 3)

In the detection device 100 of the first embodiment, the cover 35 is provided on the gap 34a between the sensor substrates 32 and on the gaps 34b between the sensor substrates 32 and the lower wall portion 40c. This makes it possible to suppress the gas from flowing in or out of the storage chamber 20 through the gaps 34a and 34b. Generally, the mother substrate is provided below the sensor substrate 32, and the power supply circuit is provided on the mother substrate. Therefore, it is conceivable that the gas heated by the power source flows into the storage chamber 20, but the provision of the cover 35 can suppress the heated gas from flowing into the storage chamber 20. This makes it possible to suppress a decrease in detection sensitivity due to the influence of disturbance.

### (Measure 4)

In the detection device 100 of the first embodiment, the cover 35 is provided so as to cover the upper surface of the sensor substrate 32. If the cover 35 is made of stainless steel, for example, gas molecules are less likely to be adsorbed on the surface of the cover 35 exposed in the space 45 than on the upper surface of the sensor substrate 32. This can suppress a decrease in the detection accuracy.

### Second Embodiment

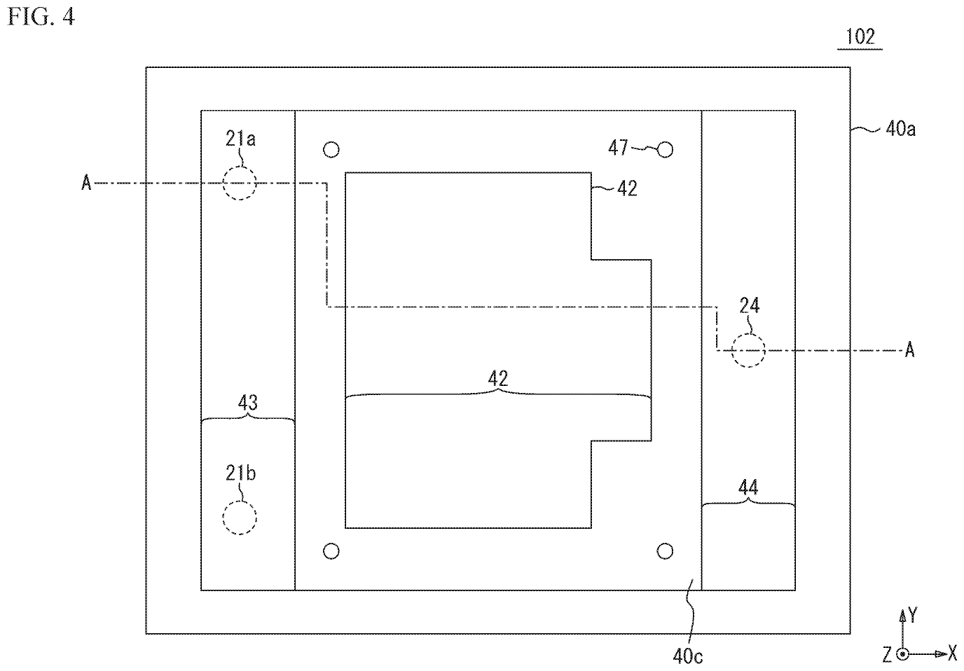

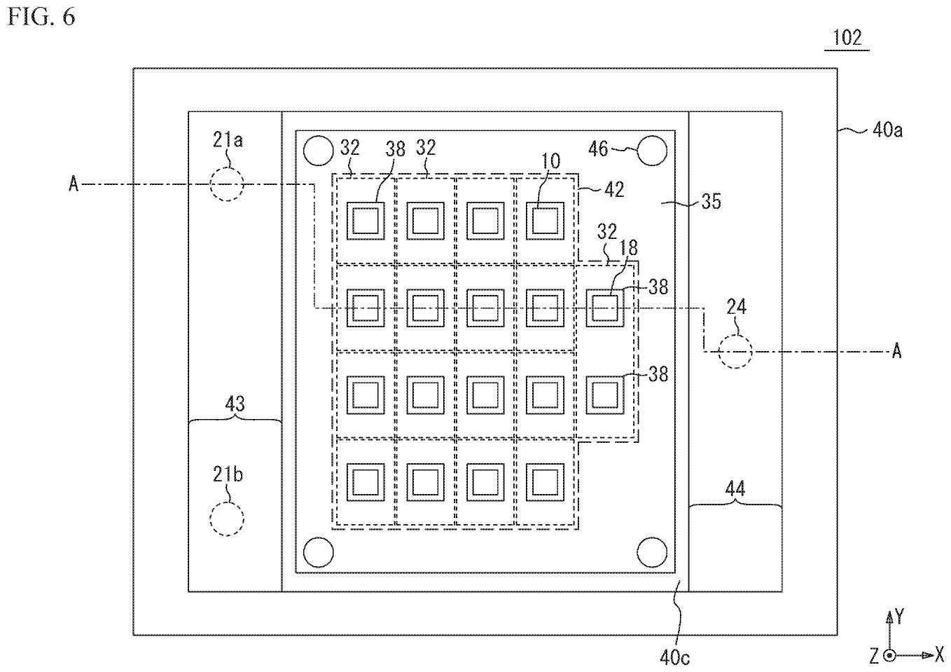

A second embodiment is a specific example of the first embodiment. FIG. 3 is a cross-sectional view of a detection device 102, and FIGS. 4 to 6 are plan views of the detection device 102 according to the second embodiment. FIG. 3 corresponds to an A-A cross section of FIGS. 4 to 6. FIG. 4 is a plan view of a housing 40a as viewed from above. FIG. 5 is a plan view of a state in which the sensor substrates 32 is disposed in the opening 42 of the lower wall portion 40c as viewed from above. FIG. 6 is a plan view of a state in which the cover 35 is disposed on the sensor substrates 32 with as viewed from above. A thickness direction of the sensor substrate 32 is referred to as a Z direction, and arrangement directions of the sensor substrates 32 are referred to as an X direction and a Y direction.

As illustrated in FIGS. 3 to 6, the housing 40 includes housings 40a and 40b. A lower portion of the housing 40a under the storage chamber 20 is the lower wall portion 40c. The housings 40a and 40b are joined by screws or the like, for example. The space 45 is formed by the housings 40a and 40b, the sensor substrates 32, the sensors 10, and the cover 35. The inner surfaces of the housing 40 surrounding the space 45 form the storage chamber 20. The lower wall portion 40c of the housing 40a is provided with the opening 42 and recesses 43 and 44 adjacent to the opening 42 in the ±X direction. The plurality of sensor substrates 32 are arranged in the opening 42 in the X direction and the Y direction in FIG. 3. The sensors 10 are provided on the upper surfaces of the sensor substrates 32. The sensor 18 is provided on the upper surface of the sensor substrate 32 located at an end in the ±X direction. The sensor 10 is a sensor that detects one or more specific substances (molecule or the like) in the gas. The sensor 18 is a sensor that detects indicators related to an environment such as temperature, humidity and pressure of the gas.

The plurality of sensor substrates 32 are laid in the opening 42 when viewed in a plan view, and the upper surfaces of the sensor substrates 32 forms at least a part of the lower surface of the storage chamber 20. The pins 33 electrically connected to the sensor 10 are buried under the sensor substrate 32. The circuit substrate 31 (mother substrate) is provided below the housing 40a. The lower surface of the housing 40a and the circuit substrate 31 are joined by a joint portion 41. The pins 33 are detachably attached to the circuit substrate 31. The pins 33 can be pulled out from the circuit substrate 31 and attached or detached. The pins 33 may be pulled out from the sensor substrate 32. A connector may be attached to the back surface of the sensor substrate 32, so that the connector is detachably attachable to another connector formed on the circuit substrate 31. This enables the sensor substrates 32 to be individually replaced, and only the sensor substrate 32 on which the deteriorated or failed sensor 10 is mounted can be replaced, thereby providing excellent cost performance.

The cover 35 is provided so as to cover the sensor substrate 32. The cover 35 includes a lower sheet-like layer 35a and a layer 35b which is provided on the layer 35a and is an original cover. For example, the cover 35 has a structure in which an insulating heat conductive sheet is bonded to the back surface of a metal plate made of stainless steel. The layer 35a brings the lower surface of the layer 35b (e.g., a metal plate) into contact with the upper surface of the sensor substrate 32, and brings the lower surface of the layer 35b into contact with the upper surface of the lower wall portion 40c. The layer 35a may be formed of a packing. The layer 35b is preferably a layer made of a material having a high thermal conductivity. As illustrated in FIG. 4, screw holes 47 are provided in the upper surface of the lower wall portion 40c. The cover 35 can be attached by inserting screws from the upper surface of the cover 35 into the screw holes 47 and joining the screws.

The materials of the housings 40a and 40b are insulators such as a fluorinee-based resin such as PTFE (polytetrafluoroethylene) and PFA (perfluoroalkoxyalkane) or other resins. The materials of the housings 40a and 40b may be metal. The layer 35b of the cover 35 is a metal plate made of stainless steel, aluminum, copper, or the like. The layer 35b may be the fluorine-based resin such as PTFE or PFA. The layer 35b is preferably made of a material having a high thermal conductivity and hardly adsorbing gas molecules to the surface. When the layer 35a is used as a heat conductive sheet, the layer 35a is, for example, a silicon heat radiation sheet. When the layer 35a is used as a packing, the layer 35a is a resin foam such as polypropylene foam. The insulating layer of the sensor substrate 32 is, for example, a glass epoxy resin. The pin 33 has conductivity, is electrically connected to the sensor 10 and the conductor pattern of the sensor substrate 32, and is buried in the through hole of the sensor substrate 32. The pin 33 is made of, for example, copper.

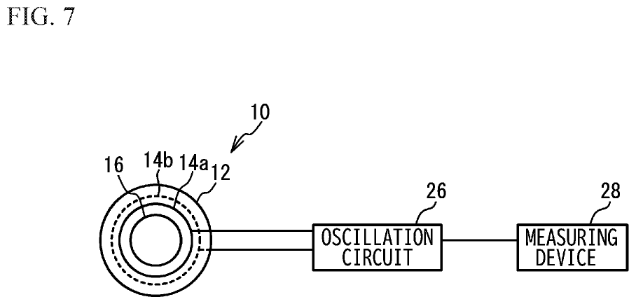

FIG. 7 is a schematic view of the sensor 10 according to the second embodiment. A quartz crystal microbalance (QCM) using a crystal resonator as the sensor 10 will be explained as an example. The sensor 10 includes a crystal plate 12 and electrodes 14a and 14b that sandwich the crystal plate 12. The sensitive film 16 is provided on the electrode 14a. The electrodes 14a and 14b are electrically connected to an oscillation circuit 26. The oscillation circuit 26 oscillates at an oscillation frequency related to a resonance frequency of the sensor 10. A measuring device 28 measures the oscillation frequency as a detected value related to the resonance frequency of the sensor 10.

The crystal plate 12 is a single crystal quartz, for example, an AT-cut crystal substrate. The electrodes 14a and 14b are metal layers containing a metal such as gold or copper as a main component.

The material of the sensitive film 16 is, for example, a polymer material, a porous material, or an organometallic compound. Examples of the polymer material include cellulose, a fluorine-based polymer, polyethyleneimine, an ester-based polymer, an acrylic polymer, polystyrene, polybutadiene, and a cycloolefin polymer, and the polymer material has a functional group to which one or more specific substances are easily bonded. The porous material is, for example, zeolite, Metal Organic Framework (MOF) such as UiO-66 or ZIF-8. The organometallic compound is, for example, a metal phthalocyanine or a metal porphyrin. The metal of the organometallic compound is, for example, copper, nickel, cobalt or zinc.

When molecules of one or more specific substances in the gas are adsorbed on the sensitive film 16, the mass of the sensitive film 16 increases. This lowers the resonance frequency of the sensor 10, and thus lowers the oscillation frequency. The crystal resonator preferably has a surface mounting type structure in which one electrode 14a faces the upper surface and the sensitive film 16 is provided on the electrode 14a. With this structure, the sensitive film 16 can be exposed to the opening 38 of the cover 35. The plurality of sensors 10 includes the sensitive films 16 made of materials different from each other. This allows each sensor 10 to differ in the amount of gas adsorbed or desorbed by the sensitive film 16 when the gas containing a single substance or complex substances of a certain concentration is supplied.

As the sensor 10, a resonator using a piezoelectric layer, such as a surface acoustic wave (SAW) resonator or a bulk acoustic wave (BAW) resonator such as a film bulk acoustic resonator (FBAR) or a solidly mounted resonator (SMR), can be used in addition to the crystal resonator. It is preferable to use surface-mount type sensors for these sensors 10, and it is preferable that the sensitive film 16 is provided flatly on the uppermost surface of the sensor 10. The sensitive film 16 is exposed from the opening 38 of the cover 35. One or more substances in the gas to be detected are, for example, an organic compound such as ethanol, acetone or toluene, or an inorganic substance such as ammonia, nitrogen oxide, ozone or chlorine.

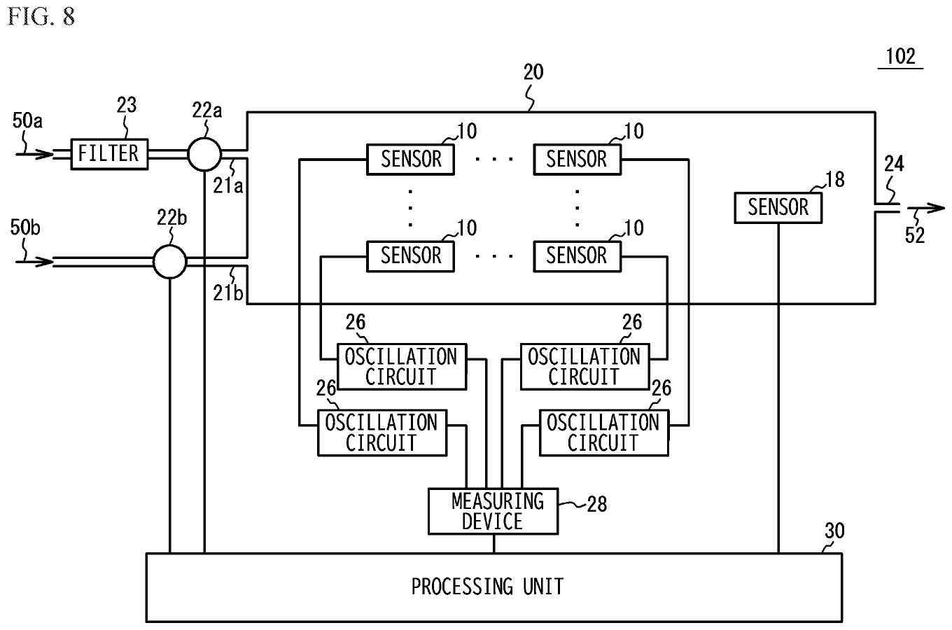

FIG. 8 is a block diagram of the detection device 102 according to the second embodiment. The sensors 10 serving as the odor sensors and the sensor 18 serving as the temperature sensor and/or the humidity sensor (environment sensor) are provided in the storage chamber 20. The plurality of sensors 10 serving as the odor sensors are connected to a plurality of oscillation circuits 26, respectively. The measuring device 28 measures the oscillation frequencies of the plurality of oscillation circuits 26.

Introduction paths 21a and 21b are provided on the left side of the storage chamber 20, and gases 50a and 50b are introduced into the storage chamber 20 from the introduction paths 21a and 21b, respectively. The discharge path 24 is provided on the right side of the storage chamber 20, and the dry and clean gas 52 that has passed through the storage chamber 20 is discharged from the discharge path 24. Pumps 22a and 22b are provided in the introduction paths 21a and 21b, respectively. By driving the pump 22a, the gas 50a as a reference is introduced into the storage chamber 20. For this reason, a filter 23 is attached to the front or rear of the pump 22a.

The reference gas 50a is dry and clean air with reduced moisture (humidity) and reduced odor components (molecules of one or more specific substances). For example, by passing the outside gas through the filter 23, moisture and odor components can be removed. By driving the pump 22b, the gas 50b to be detected is introduced into the storage chamber 20. The gas 50b is, for example, air containing the odor component.

A processing unit 30 is, for example, a processor. Software is incorporated in the processing unit 30. The processing unit 30 calculates or determines information about the gas 50b based on information about the resonance frequencies of the sensors 10 output from the measuring device 28 and the temperature, the pressure and the like output from the sensor 18. The information about the gas 50b is, for example, information about the concentration of the one or more specific substances in the gas 50b or information about the odor component contained in the gas 50b. The processing unit 30 controls the pumps 22a and 22b. At least a part of the processing unit 30 may be formed by hardware such as a dedicated circuit.

For example, a correspondence relationship between information corresponding to changes in the resonance frequencies in the sensors 10 caused by changes in temperature or humidity and the output of the sensor 18 which is the temperature sensor or the humidity sensor is stored in a memory. The processing unit 30 can correct the disturbance of the temperature or the humidity from the information about the resonance frequencies of the sensors 10 based on the output of the sensor 18 and the above-mentioned correspondence relationship. For this reason, it is more preferable that the temperature or humidity around the plurality of sensors 10 and 18 is uniform as much as possible. Therefore, the cover 35 is in contact with a part of the upper surface of each of the plurality of sensor substrates 32 on which the plurality of sensors 10 are mounted and a part of the upper surface of the sensor substrate 32 (environment sensor substrate) on which the sensor 18 (environment sensor) is mounted, and the openings 38 expose the plurality of sensors 10 and the sensor 18. This makes it possible to make the temperature or humidity around the plurality of sensors 10 and the sensor 18 more uniform.

### [Experiment 1]

The temperatures of the sensors 10 and 18 were measured with an infrared sensor, using the second embodiment with the cover 35 and a second comparative example without the cover 35. The layer 35b of the cover 35 used in the experiment was a stainless steel plate having a thickness of 0.5 mm, and the layer 35a was a polypropylene foam.

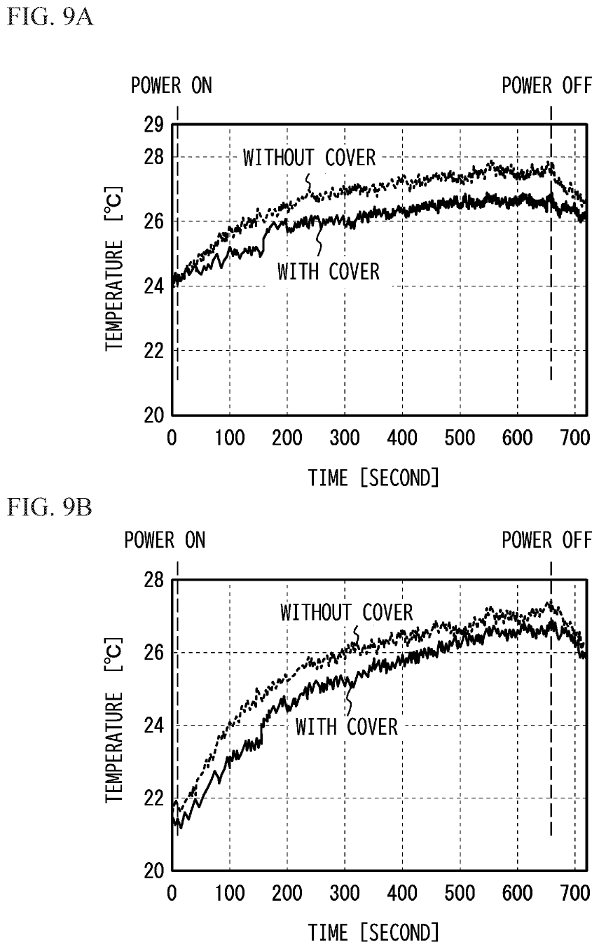

FIGS. 9A and 9B are diagrams illustrating the temperatures of the sensors with respect to time in Experiment 1. FIG. 9A illustrates the temperature of a sensor 10a at an upper left end (−X and +Y side) of FIG. 5, and FIG. 9B illustrates the temperature of the sensor 18a (temperature sensor) at a lower right end. Power On represents a time when the power of the sensors 10 and 18 is turned on, and Power Off represents a time when the power of the sensors 10 and 18 is turned off. The “Power On” means that the power is supplied to the sensor 10 and the peripheral circuits of the sensor 10, a control signal is sent, and measurement is started. The measurement is performed over time, for example, by oscillating the crystal resonator using the oscillation circuit 26 and counting the oscillation frequency by a frequency counter.

The temperatures of the sensors 10a and 18a rise when the power is turned on, and the temperatures of the sensors 10a and 18a fall when the power is turned off. The temperature of the sensors 10a and 18a in the second embodiment with the cover 35 increases less than that in the second comparative example without the cover 35. This is thought to be because, in the second embodiment, heat generated in the sensors 10a and 18a is efficiently conducted to the housing 40a through the cover 35, and heat is released from the housing 40a. This is also thought to be because the cover 35 functions as a heat sink.

### [Experiment 2]

Variations in sensitivity of the plurality of sensors 10 were measured using the second embodiment with the cover 35 and a second comparative example without the cover 35. The sensitive films 16 of the upper (+Y side) eight sensors 10 in FIG. 5 were made the same, and the sensitivity of each sensor 10 was measured. The layer 35b of the cover 35 used in the experiment was the aluminum plate having the thickness of 0.5 mm, and the layer 35a was the polypropylene foam. At a temperature of 24° C., clean and dry air as a reference was introduced into the storage chamber 20, and the resonance frequencies fr0 of the plurality of sensors 10 were measured, and then air containing toluene at a concentration of 50 ppm was introduced into the storage chamber 20, and the resonance frequencies fr of the plurality of sensors 10 were measured. The sensitivity is defined as |fr−fr0| in each sensor 10. The same operation was repeated three times, and the sensitivity was measured three times.

Table 1 represents a maximum, a minimum and a difference of sensitivity in the second comparative example without the cover 35.

TABLE 1

| | MAXIMUM | MINIMUM | DIFFERENCE |
|---|---|---|---|
| | [Hz] | [Hz] | [Hz] |
| 1ST TIME | 430 | 376 | 54 |
| 2ND TIME | 428 | 378 | 50 |
| 3RD TIME | 432 | 378 | 54 |
| AVERAGE | 430 | 377 | 53 |

Table 2 represents a maximum, a minimum and a difference of sensitivity in the second embodiment with the cover 35.

TABLE 2

| | MAXIMUM | MINIMUM | DIFFERENCE |
|---|---|---|---|
| | [Hz] | [Hz] | [Hz] |
| 1ST TIME | 422 | 378 | 44 |
| 2ND TIME | 424 | 383 | 41 |
| 3RD TIME | 426 | 384 | 42 |
| AVERAGE | 424 | 382 | 42 |

In Tables 1 and 2, “maximum”, “minimum”, and “difference” indicate a maximum sensitivity, a minimum sensitivity, and a difference between the maximum and minimum sensitivities of the eight sensors 10, respectively. As illustrated in Table 1, in the second comparative example, the difference between the maximum and minimum is 50 to 54 Hz, and the average of the difference is 53 Hz. As illustrated in Table 2, in the second embodiment, the difference between the maximum and minimum is 41 to 44 Hz, and the average of the difference is 42 Hz.

As described above, in the second embodiment, the variations in sensitivity of the plurality of sensors 10 are smaller than those in the second comparative example. This is thought to be because the temperature variation between the sensors 10 in the second embodiment is smaller than that in the second comparative example due to the cover 35.

In the experiments 1 and 2, polypropylene foam assumed to be a packing was used as the layer 35a of the cover 35 of the second embodiment, but it is considered that a more effective effect can be obtained by using a silicon heat radiation sheet or the like having a high thermal conductivity as the layer 35a.

If the thermal conductivity of the layer 35b is low, the conduction of heat through the cover 35 is reduced. From this viewpoint, it is preferable that the thermal conductivity of at least one layer (e.g., the layer 35b) in the cover 35 is higher than the thermal conductivity of the insulating layer in the sensor substrate 32. For example, the thermal conductivity of FR-4 used as the sensor substrate 32 is about 0.3 W/(m·K), the thermal conductivity of stainless steel is about 16 W/(m·K), and the thermal conductivity of aluminum is 200 W/(m·K). The thermal conductivity of at least one layer (e.g., layer 35b) of the cover 35 is preferably at least twice the thermal conductivity of the insulating layer in the sensor substrate 32, and more preferably at least ten times the thermal conductivity of the insulating layer in the sensor substrate 32.

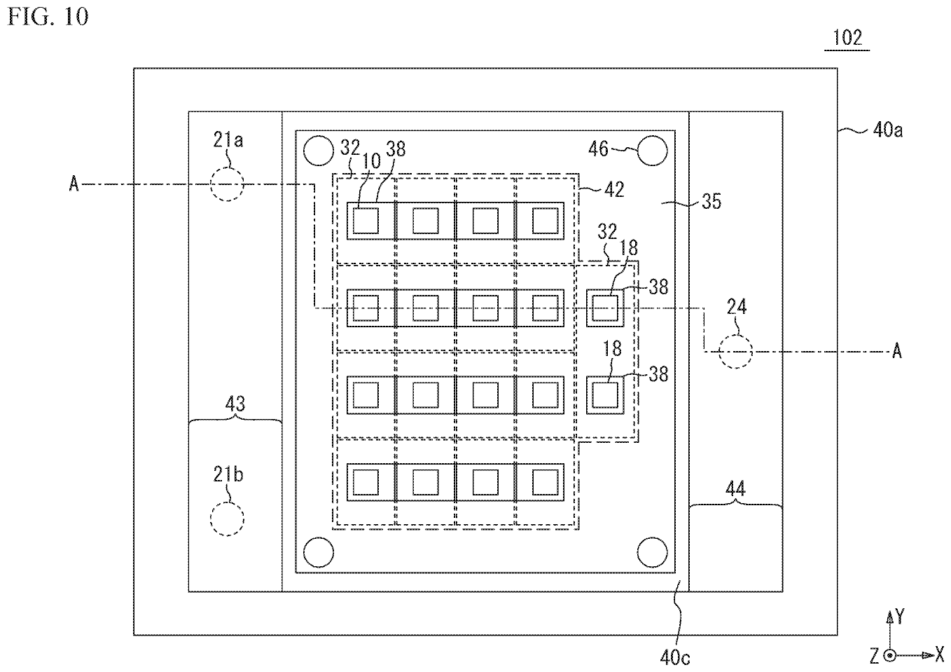

FIG. 10 is a plan view of another example of the detection device according to the second embodiment, and is a plan view of a state in which the cover 35 is disposed on the sensor substrate 32 as viewed from above. As illustrated in FIG. 10, the plurality of sensors 10 may be provided in one opening 38 of the cover 35. The opening 38 of the cover 35 is preferably provided for each set including the plurality of sensors 10.

The number of the sensor substrates 32 may be one, and the plurality of sensors 10 may be mounted on one sensor substrate 32. The number of sensor substrates 32 may be plural, and one or the plurality of sensors 10 may be mounted on each of the plurality of sensor substrates 32.

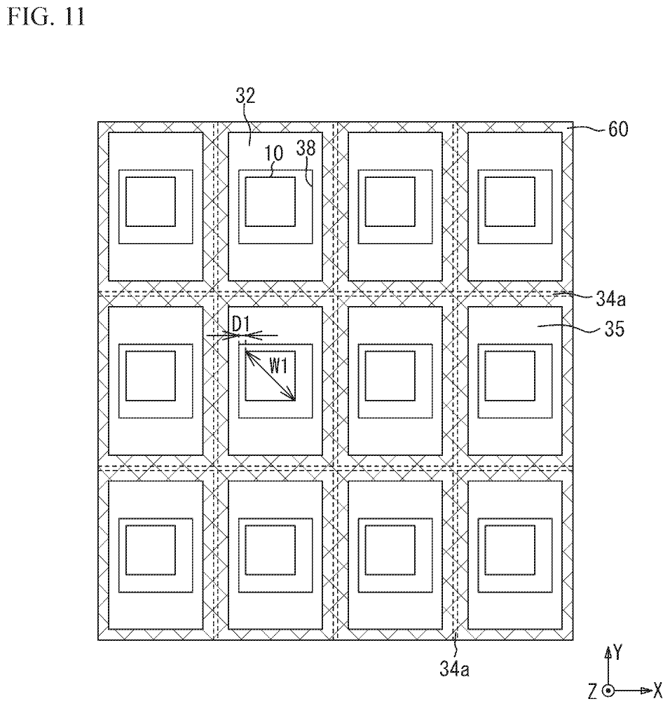

FIG. 11 is an enlarged plan view of the detection device according to the second embodiment. The cover 35 is preferably in contact with a peripheral edge region like a region 60 of the upper surface of each of the sensor substrates 32 to surround the sensor 10. As a result, heat is conducted from the sensor substrate 32 to the adjacent sensor substrates 32 via the cover 35 while bypassing the gap 34a between the sensor substrates 32. This makes it possible to suppress the temperature variations between the sensors 10. Further, the leakage and inflow of the gas through the gap 34a can be suppressed. In a plan view, it is preferable that most of the cover 35 is in contact with the sensor substrates 32 and the housing 40a.

If a shortest distance D1 between the side surface of the opening 38 of the cover 35 and each of the plurality of sensors 10 is too large, heat is less likely to be conducted through the gas between the sensor 10 and the cover 35. This increases the temperature variations between the sensors 10. In addition, an area of the surface of the sensor substrate 32 exposed from the opening 38 becomes large. This increases the size of the specific gas molecules adsorbed on the surface of the sensor substrate 32. From this viewpoint, the distance D1 is preferably equal to or less than a maximum width W1 of the sensor 10, more preferably equal to or less than 0.5 times the maximum width W1, and even more preferably equal to or less than 0.2 times the maximum width W1.

When the sensor 10 is configured to provide a piezoelectric body and electrodes sandwiching the piezoelectric body on an insulating substrate such as silicon, the silicon substrate has insulating properties, and therefore the sensor 10 can be brought into contact with the cover 35. In this manner, the configuration in which a part of the sensor 10 and the cover 35 are in contact with each other is more preferable because the sensor 10 and the cover 35 can directly exchange heat.

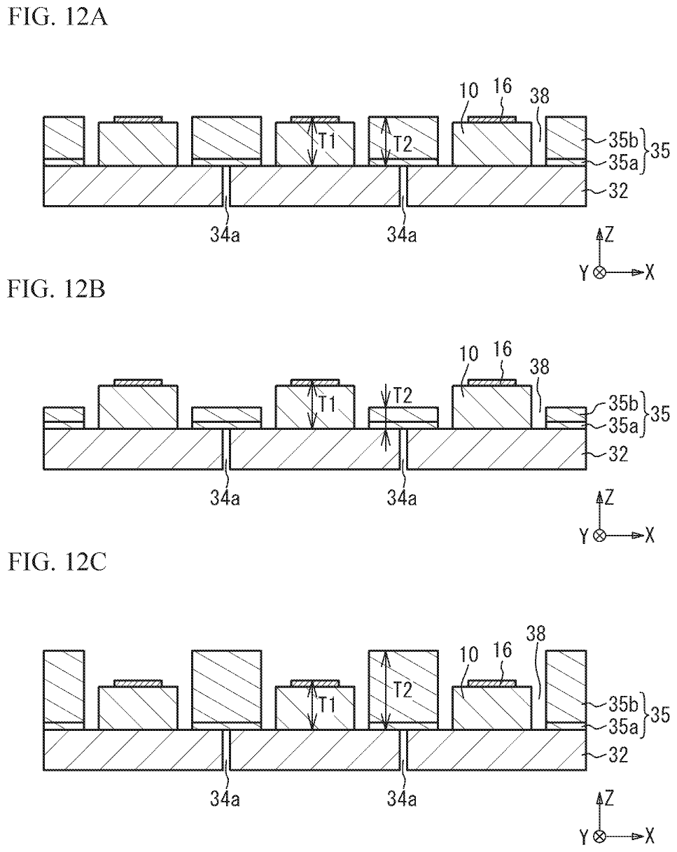

FIGS. 12A to 12C are enlarged sectional views of the detection device according to the second embodiment. In FIG. 12A, a thickness T1 of the sensor 10 and a thickness T2 of the cover 35 are substantially the same as each other. In FIG. 12B, the thickness T2 of the cover 35 is smaller than the thickness T1 of the sensor 10. In FIG. 12C, the thickness T2 of the cover 35 is larger than the thickness T1 of the sensor 10.

As illustrated in FIG. 12B, when the cover 35 is thin, heat is less likely to be conducted through the cover 35. From this viewpoint, the thickness T2 of the cover 35 is preferably ½ or more of the thickness T1 of the sensor 10, and more preferably ⅔ or more of the thickness T1 of the sensor 10.

As illustrated in FIG. 12C, if the cover 35 is thick, the gas is less likely to reach the sensitive film 16 of the sensor 10. From this viewpoint, the thickness T2 of the cover 35 is preferably twice or less the thickness T1 of the sensor 10, and more preferably 1.5 or less the thickness T1 of the sensor 10.

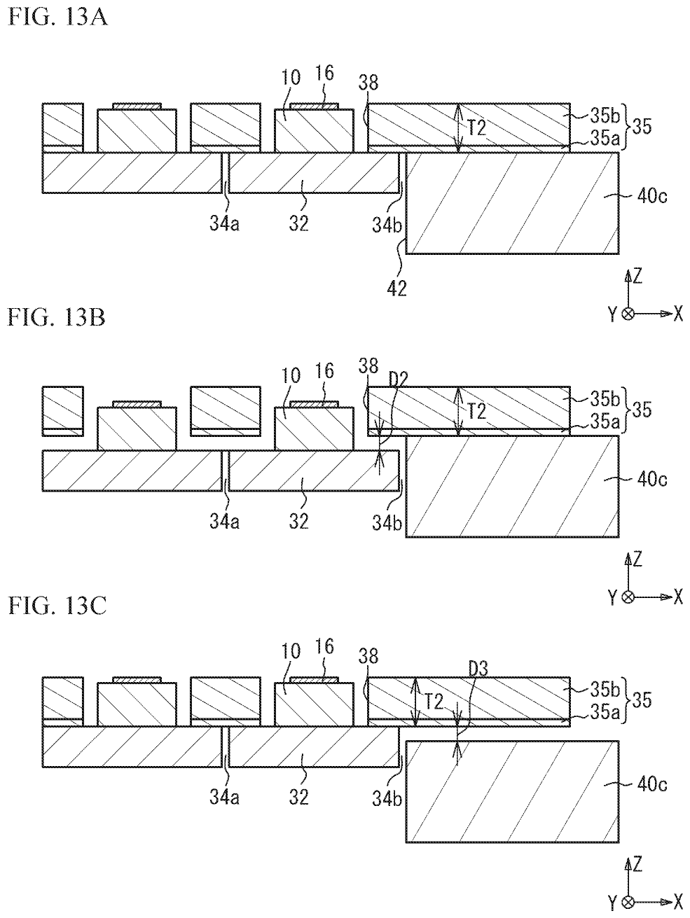

FIGS. 13A to 13C are enlarged sectional views of the detection device according to the second embodiment. In FIG. 13A, the height of the upper surface of the sensor substrate 32 is substantially the same as the height of the upper surface of the lower wall portion 40c of the housing 40. In FIG. 13B, the upper surface of the sensor substrate 32 is lower than the upper surface of the lower wall portion 40c of the housing 40. A difference in height between the upper surface of the sensor substrate 32 and the upper surface of the lower wall portion 40c is D2. In FIG. 13C, the upper surface of the sensor substrate 32 is higher than the upper surface of the lower wall portion 40c of the housing 40. A difference in height between the upper surface of the sensor substrate 32 and the upper surface of the lower wall portion 40c is D3.

As illustrated in FIG. 13B, when the upper surface of the sensor substrate 32 is lower than the upper surface of the lower wall portion 40c of the housing 40, the cover 35 is less likely to contact the sensor substrate 32 when the cover 35 is attached to the lower wall portion 40c by using mounting members such as screws 46 as illustrated in FIG. 6. This makes it difficult to conduct heat between the sensor substrates 32. Further, the gas leaks and enters through the gap 34a.

As illustrated in FIG. 13C, when the upper surface of the sensor substrate 32 is higher than the upper surface of the lower wall portion 40c of the housing 40, the cover 35 is less likely to contact the lower wall portion 40c when the cover 35 is attached using the screws 46. This makes it difficult to conduct heat between the sensor substrate 32 and the lower wall portion 40c. Further, the gas leaks and enters through the gap 34b.

When the cover 35 is attached to the lower wall portion 40c by using the screws 46 (mounting members) and the screw holes 47, if the differences D2 and D3 in height between the upper surface of the sensor substrate 32 and the upper surface of the lower wall portion 40c are large, the effect of the second embodiment is not sufficiently generated as illustrated in FIGS. 13B and 13C. Therefore, the differences D2 and D3 are preferably 1/10 or less of the thickness T2 of the cover 35, and more preferably 1/20 or less of the thickness T2 of the cover 35.

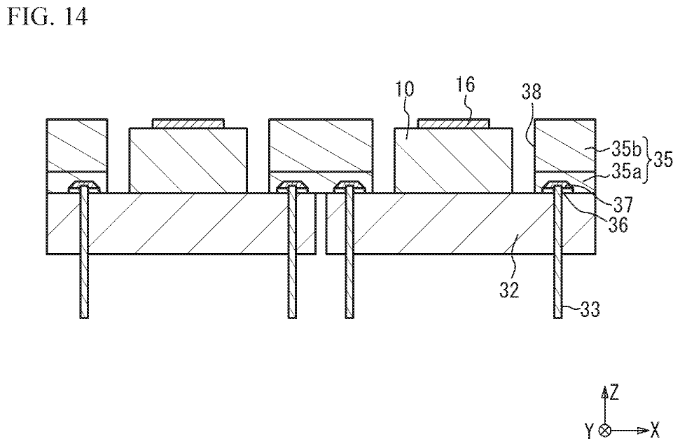

FIG. 14 is an enlarged cross-sectional view of the detection device according to the second embodiment. Pads 36 are provided on the upper surface of the sensor substrate 32. The pad 36 is provided around at least one of the plurality of sensors 10 or an element (e.g., an element constituting a part of the oscillation circuit 26) constituting a part of a circuit (e.g., a peripheral circuit) electrically connected to at least one of the plurality of sensors 10. The circuit is, for example, the oscillation circuit 26 electrically connected to the sensor 10 or a power supply circuit of the oscillation circuit 26. The element constituting the part of the circuit is an individual component such as a resistor, a capacitor, a inductor or a transistor, or an integrated circuit. The element is, for example, an integrated circuit provided on the lower surface of the crystal resonator of the sensor 10, and may constitute a module together with the sensor 10. The element may be provided on the lower surface of the sensor substrate 32. Further, the element may be provided on the upper surface of the sensor substrate 32 so as to be exposed in the opening 38 of the cover 35. Further, the element may be provided in contact with the cover 35 in the upper surface of the sensor substrate 32. In this case, if the layer 35a of the cover 35 is a packing made of, for example, an insulating resin, the layer 35a presses the element so that a gap between the layer 35a and the element can be reduced. This allows the cover 35 to contact the sensor substrate 32 over a large area.

The pin 33 extends through the sensor substrate 32 and the pad 36. The pin 33 and the pad 36 are bonded by a conductive bonding portion 37 such as a solder. The pin 33 may not be electrically connected to the pad 36 on the upper surface of the sensor substrate 32, but may be electrically connected to a pad formed on the lower surface of the sensor substrate 32. A through hole extending through the sensor substrate 32 may be provided, and the pad 36 and the pad on the lower surface of the sensor substrate 32 may be electrically connected to each other through the through hole. Further, a wiring electrically connected to the sensor 10 or the peripheral circuit of the sensor 10 may be provided on the upper surface of the sensor substrate 32.

When the pad 36, the bonding portion 37, and a conductor pattern such as the wiring are provided on the upper surface of the sensor substrate 32, it is preferable that at least the lower surface of the cover 35 has an insulation property. This can suppress the pads 36 or the wirings of the adjacent sensor substrates 32 from being electrically short-circuited to each other via the cover 35. For example, by using the layer 35a made of an insulator, electrical short-circuiting between the pads 36 or the wirings of the adjacent sensor substrates 32 can be suppressed.

Although FIG. 14 does not necessarily accurately reflect the scale of the actual product, an area of the pad 36 occupies a large proportion of the upper surface of the sensor substrate 32 in a plan view. Although the wiring is not illustrated, the wiring is sufficiently small in scale as compared to the pad 36 or pin 33. It can be understood that the pad 36 is large enough in scale to contribute to heat conduction, as compared with this wiring. Therefore, when the pad 36 or the bonding portion 37 formed on the pad 36 is into contact with the cover 35, heat generated by the sensor 10 or the peripheral circuit of the sensor 10 can be efficiently transmitted to the cover 35.

Further, it is preferable that a plurality of pads 36 are provided in a peripheral edge region of the upper surface of the sensor substrate 32 and are in contact with the cover 35. In the above embodiments, the plurality of sensor substrates 32 are arranged in a matrix form in a plan view, with the side surfaces of the sensor substrates 32 touching each other. If the plurality of pads 36 are provided in the peripheral edge region of the upper surface of each of the plurality of sensor substrates 32, the plurality of pads 36 of two adjacent sensor substrates 32 are arranged so that they are lined up next to each other in two rows. Since the plurality of pads 36 are in contact with the cover 35, the heat transfer property can be improved.

Although the embodiments of the present disclosure have been described in detail, it is to be understood that the various change, substitutions, and alterations could be made hereto without departing from the spirit and scope of the invention.

What is claimed is:

1. A detection device comprising:
a plurality of sensors each of which is configured to detect one or more substances causing an odor in a gas;
a storage chamber that stores the plurality of sensors;
one or more sensor substrates each forming at least a part of a lower surface of the storage chamber and each having an upper surface on which one or more of the plurality of sensors are mounted; and
a cover provided on the one or more of sensor substrate, having one or more openings that expose the plurality of sensors, and being in contact with the upper surface of the one or more sensor substrates.

2. The detection device according to claim 1, wherein:
a plurality of sensor substrates are provided,
the plurality of sensor substrates are adjacent to each other, and
the cover is in contact with the upper surface of each of the plurality of sensor substrates.

3. The detection device according to claim 2, wherein
the cover is provided on a peripheral edge region of the upper surface of each of the plurality of sensor substrates.

4. The detection device according to claim 2, further comprising:
a circuit substrate electrically connected to the plurality of sensor substrates and being provided below the plurality of sensor substrates,
wherein the plurality of sensor substrates are detachably attachable to the circuit substrate.

5. The detection device according to claim 1, wherein at least some of the one or more openings expose two or more of the plurality of sensors.

6. The detection device according to claim 5, wherein
a shortest distance between each of the plurality of sensors and a side surface of the opening is smaller than a maximum width of each of the plurality of sensor.

7. The detection device according to claim 1, wherein:
a conductor pattern is provided on the upper surface of the one or more sensor substrates, and
at least a lower surface of the cover has an insulation property, or an insulating layer is provided between the cover and the conductor pattern.

8. The detection device according to claim 7, wherein the conductor pattern is a pad provided around at least one of the plurality of sensors or an element constituting a part of a circuit electrically connected to the at least one of the plurality of sensors.

9. The detection device according to claim 1, wherein a thermal conductivity of at least a part of layers in the cover is higher than a thermal conductivity of an insulating layer in the one or more sensor substrates.

10. The detection device according to claim 1,
wherein the storage chamber is defined by a housing, the one or more sensor substrates and the cover, the housing having a lower wall portion an upper surface of which forms a part of the lower surface of the storage chamber, the lower wall portion of the housing having a cavity, the one or more sensor substrates being provided inside the cavity in a plan view, and the cover is mounted to the lower wall portion via a mounting member, and
wherein a difference in height between the upper surface of the one or more sensor substrates and the upper surface of the lower wall portion is 1/10 or less of a thickness of the cover.

11. The detection device according to claim 1, wherein
a thickness of the cover is ½ times or more and twice or less a thickness of the plurality of sensors.

12. The detection device according to claim 2, further comprising:
an environment sensor substrate having an environment sensor provided on an upper surface thereof, the environment sensor being a temperature sensor or a humidity sensor;
wherein the cover is in contact with the upper surface of each of the plurality of sensor substrates and the upper surface of the environment sensor substrate, and the one or more openings expose the plurality of sensors and the environment sensor.
